(19) United States
(12) Patent Application Publication
(10) Pub. No.: US 2024/0350535 A1
(43) Pub. Date: Oct. 24, 2024

(54) METHOD OF TREATING CANCER USING (TRI-TERT-BUTYLPHOSPHANE)GOLD(I) THIONE COMPLEXES

(71) Applicant: KING FAHD UNIVERSITY OF PETROLEUM AND MINERALS, Dhahran (SA)

(72) Inventors: Adam Ahmed Abdallah SULAIMAN, Dhahran (SA); Anvarhussien A. ISAB, Dhahran (SA); Ali R. ALHOSHANI, Dhahran (SA)

(73) Assignee: KING FAHD UNIVERSITY OF PETROLEUM AND MINERALS, Dhahran (SA)

(21) Appl. No.: 18/303,910

(22) Filed: Apr. 20, 2023

(51) Int. Cl. A61K 33/242; A61P 35/04

(52) U.S. Cl. CPC A61K 33/242; A61P 35/04

(57) ABSTRACT

A method for treating a cancer including administering to a subject in need thereof a complex of formula (I), where R¹ is a substituted or unsubstituted cyclic amine, a ring of the cyclic amine has 5-7 atoms, R² is a straight or branched aliphatic carbon chain including 3-10 carbon atoms, and X is a counter ion.

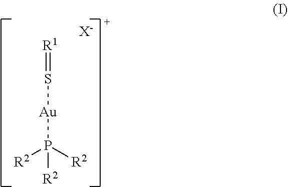

### STATEMENT OF ACKNOWLEDGEMENT

Support provided by King Fahd University of Petroleum and Minerals (KFUPM) under grant number INAM2210 is gratefully acknowledged.

### BACKGROUND

### Technical Field

The present disclosure is directed to metal complexes, more particularly (tri-tert-butylphosphane)gold(I) thione complexes, and to a method of treating cancer using (tri-tert-butylphosphane)gold(I) thione complexes.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Metal complexes are used in a variety of pharmaceutical applications, for example, auranofin is a linear Au(I) complex of [2,3,4,6-tetra-o-acetyl-L-thio-b-D-glycopyranp-sato-S-(tri-ethyl-phosphine)-gold] approved for treatment of rheumatoid arthritis. Also, Auranofin was clinically studied as an anticancer drug and for other diseases, such as Trichomonas vaginalis and craniofacial defects. Therefore, research on gold complexes containing phosphine and sulfur donor ligands for pharmaceutical applications, especially in cancer chemotherapy, has increased.

Although the mechanism of action of gold complexes is not fully understood, several compounds have been identified to cause cell death by inhibiting the reduction/oxidation of the activity of various enzymes, such as TrxR and lymphoid tyrosine phosphatase. Enzyme inhibitors induce cancer cell death via excessive oxidative stress that causes cellular oxidative stress, intrinsic apoptosis, and mitochondria dysfunction.

Although gold complexes for cancer treatment have been developed in the past, there is still a need for efficient complexes which may show a high degree of selectivity towards cancerous cells at low concentrations.

### SUMMARY

In an exemplary embodiment, a method for treating a cancer is described. The method includes administering to a subject in need thereof a complex of formula (I),

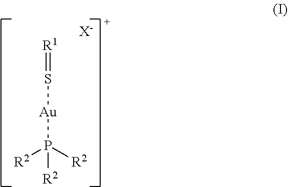

where R¹ is a substituted or unsubstituted cyclic amine, a ring of the cyclic amine has 5-7 atoms, R² is a straight or branched aliphatic carbon chain including 3-10 carbon atoms, and X is a counter ion.

In some embodiments, the ring of the cyclic amine has 6 atoms and has the structure of formula (II),

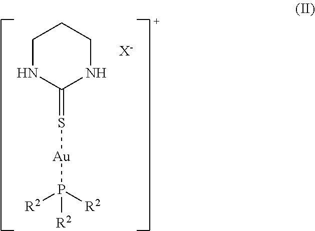

In some embodiments, the ring of the cyclic amine has 7 atoms and has the structure of formula (III),

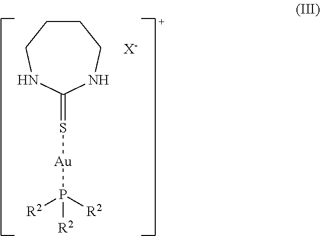

In some embodiments, the cyclic amine is substituted with a substituted or unsubstituted aliphatic chain including 1-10 carbon atoms at a nitrogen position of the cyclic amine.

In some embodiments, the cyclic amine is substituted with an unsubstituted aliphatic chain including 2 carbon atoms at a nitrogen position of the cyclic amine and has a structure of formula (IV),

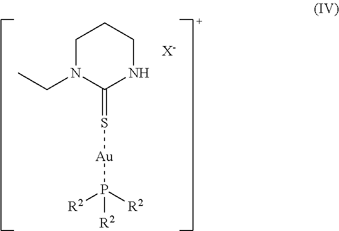

In some embodiments, the cyclic amine is substituted with a substituted aliphatic chain including 2 carbon atoms and a terminal alcohol at a nitrogen position of the cyclic amine and has a structure of formula (V),

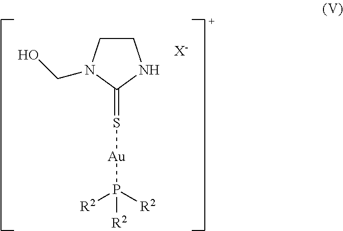

In some embodiments, the counter ion is selected from the group consisting of fluoride, chloride, iodide, triflate, and hexafluorophosphate (PF₆⁻).

In some embodiments, R² is a tert-butyl group.

In some embodiments, the cancer is cervical cancer, osteosarcoma, or colon cancer.

In some embodiments, the complex has an IC₅₀ of 1-15 micromolar (μM) against colon cancer cells in the subject.

In some embodiments, the complex has an IC₅₀ of 3-40 μM against cervical cancer cells in the subject.

In some embodiments, the complex has an IC₅₀ of 0.1-15 μM against osteosarcoma cancer cells in the subject.

In some embodiments, following administration, the amount of colon cancer cells in the subject are reduced by at least 50%.

In some embodiments, following administration, the amount of cervical cancer cells in the subject are reduced by at least 40%.

In some embodiments, following administration, the amount of osteosarcoma cancer cells in the subject are reduced by at least 60%.

In some embodiments, the method includes administering 1-20 milligrams (mg) of the complex per kilogram (kg) of the subject.

In some embodiments, the method includes administering the complex in a subject by oral administration, parenteral administration, topical application, trans-dermally or nasally.

In some embodiments, the complex is 5-30 times more effective at reducing a number of cancer cells in the subject than cisplatin.

In some embodiments, following administration, the amount of colon cancer cells or osteosarcoma cancer cells in the subject are reduced by at least 80%.

The foregoing general description of the illustrative present disclosure and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 depicts schematic structures of complexes (0-4), i.e., chlorido(tri-tert-butylphosphine)gold(I) (0), Au(P(t-Bu)₃)(thione)](PF₆), {where, P(t-Bu)₃=tri-tert-butylphosphine and thione=diazinane-2-thione, Diaz (1); N-hydroxyethyl-1,3-imidazolidine-2-thione, 2-EtOH-Imt (2); N-ethyl-1,3-diazinane-2-thione, N-Et-Diaz (3); 1,3-diazepine-2-thione, Diap (4)}, according to certain embodiments;

FIG. 2 depicts a molecular structure of the complex (3), according to certain embodiments;

FIG. 3 depicts a crystal packing of complex 3 viewed along the b-axis, according to certain embodiments;

FIG. 4 depicts a molecular structure of complex 4, according to certain embodiments;

FIG. 5 depicts crystal packing of complex 4 viewed along the b-axis, according to certain embodiments;

FIG. 6A-FIG. 6B is a graph depicting in vitro cytotoxic effect of the complexes (0, 1-4) concentrations on cell viability of HCT-15 (colon cancer) cell lines, according to certain embodiments;

FIG. 6C-FIG. 6D is a graph depicting in vitro cytotoxic effect of the complexes (0, 1-4) concentrations on cell viability of MG-63 (osteosarcoma) cell lines, according to certain embodiments;

FIG. 6E-FIG. 6F is a graph depicting in vitro cytotoxic effect of the complexes (0, 1-4) concentrations on cell viability of HeLa (human cervical cancer) cell lines, according to certain embodiments;

FIG. 7A depicts a representative reverse transcription-polymerase chain reaction (RT-PCR) showing beta-actin (ACTB), caspase 3 (CASP 3), and caspase 9 (CASP 9) gene expression in HeLa cells, according to certain embodiments;

FIG. 7B is a graph depicting percent relative intensity measurement of caspase 3 expression in HeLa cells to 3-actin following treatment with 2 or 3, according to certain embodiments;

FIG. 7C is a graph depicting percent relative intensity measurement of caspase 9 expression in HeLa cells to 3-actin following treatment with 2 or 3, according to certain embodiments;

FIG. 8A depicts RT-PCR showing β-actin, caspase 3, and caspase 9 gene expressions in MG-63 cells, according to certain embodiments;

FIG. 8B is a graph depicting percent relative intensity measurement of caspase 3 expression in MG-63 cells to 3-actin following treatment with 2 or 3, according to certain embodiments;

FIG. 8C is a graph depicting percent relative intensity measurement of caspase 9 expression in MG-63 cells to 3-actin following treatment with 2 or 3, according to certain embodiments;

FIG. 9A depicts representative RT-PCR showing β-actin, caspase 3, and caspase 9 gene expressions in the HCT-15 cells, according to certain embodiments;

FIG. 9B is a graph depicting percent relative intensity measurement of caspase 3 expression in HCT-15 cells to 3-actin following treatment with 2 or 3, according to certain embodiments;

FIG. 9C is a graph depicting percent relative intensity measurement of caspase 9 expression in HCT-15 cells to 3-actin following treatment with 2 or 3, according to certain embodiments;

FIG. 10A depicts the average tumor volume of mice with respect to time while using a control and complex 2, according to certain embodiments; and

FIG. 10B depicts the average body weight of mice with respect to time while using a control and complex 2, according to certain embodiments.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values there between.

Where a numerical limit or range is stated herein, the endpoints are included. Also, all values and subranges within a numerical limit or range are specifically included as if explicitly written out.

As used herein, the term ‘cancer’ refers to all types of cancer, neoplasm or malignant tumors found in mammals (e.g., humans), including leukemias, lymphomas, carcinomas, and sarcomas. Exemplary cancers that may be treated with the method or composition provided herein include brain cancer, glioma, glioblastoma, neuroblastoma, prostate cancer, colorectal cancer, pancreatic cancer, medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with the method provided herein include cancer of the thyroid, endocrine system, brain, breast, cervix, colon, head and neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulinoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

As used herein, the term ‘aliphatic compound’ refers to the organic compound containing hydrogen and carbon atoms that are usually linked together in chains via single, double, or triple bonds. Sometimes the chains are also in branched trains or in the form of non-aromatic structures.

As used herein, the term “alkyl” unless otherwise specified refers to both branched and straight chain aliphatic (non-aromatic) hydrocarbons which may be primary, secondary, and/or tertiary hydrocarbons typically having 1 to 32 carbon atoms (e.g., C₁, C₂, C₃, C₄, C₅, C₆, C₇, C₈, C₉, C₁₀, C₁₁, C₁₂, C₁₃, C₁₄, etc.) and specifically includes, but is not limited to, saturated alkyl groups such as methyl, ethyl, propyl, isopropyl, butyl, isobutyl, t-butyl, pentyl, isopentyl, neopentyl, hexyl, isohexyl, 3-methylpentyl, 2,2-dimethylbutyl, 2,3-dimethylbutyl, 2-ethylhexyl, heptyl, octyl, nonyl, 3,7-dimethyloctyl, decyl, undecyl, dodecyl, tridecyl, tetradecyl, pentadecyl, hexadecyl, heptadecyl, octadecyl, nonadecyl, eicosyl, guerbet-type alkyl groups (e.g., 2-methylpentyl, 2-ethylhexyl, 2-proylheptyl, 2-butyloctyl, 2-pentylnonyl, 2-hexyldecyl, 2-heptylundecyl, 2-octyldodecyl, 2-nonyltridecyl, 2-decyltetradecyl, and 2-undecylpentadecyl), as well as unsaturated alkenyl and alkynyl variants such as vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, oleyl, linoleyl, and the like.

As used herein, the term “substituted” refers to at least one hydrogen atom that is replaced with a non-hydrogen group, provided that normal valency is maintained and that the substitution results in a stable compound. When a group is noted as “optionally substituted”, the group may or may not contain non-hydrogen substituents. When present, the substituent(s) may be selected from alkyl, halo (e.g., chloro, bromo, iodo, fluoro), hydroxyl, alkoxy, oxo, alkanoyl, aryloxy, alkanoyloxy, amino (—NH₂), alkylamino (—NHalkyl), cycloalkylamino (—NHcycloalkyl), arylamino (—NHaryl), arylalkylamino (—NHarylalkyl), disubstituted amino (e.g., in which the two amino substituents are selected from alkyl, aryl or arylalkyl, including substituted variants thereof, with specific mention being made to dimethylamino), alkanoylamino, aroylamino, arylalkanoylamino, thiol, alkylthio, arylthio, arylalkylthio, alkylthiono, arylthiono, arylalkylthiono, alkylsulfonyl, arylsulfonyl, arylalkylsulfonyl, sulfonamide (e.g., —SO₂NH₂), substituted sulfonamide (e.g., —SO₂NHalkyl, —SO₂NHaryl, —SO₂NHarylalkyl, or cases where there are two substituents on one nitrogen selected from alkyl, aryl, or alkylalkyl), nitro, cyano, carboxy, unsubstituted amide (i.e. —CONH₂), substituted amide (e.g., —CONHalkyl, —CONHaryl, —CONHarylalkyl or cases where there are two substituents on one nitrogen selected from alkyl, aryl, or alkylalkyl), alkoxycarbonyl, aryl, guanidine, heterocyclyl (e.g., pyridyl, furyl, morpholinyl, pyrrolidinyl, piperazinyl, indolyl, imidazolyl, thienyl, thiazolyl, pyrrolidyl, pyrimidyl, piperidinyl, homopiperazinyl), and mixtures thereof. The substituents may themselves be optionally substituted and may be either unprotected, or protected as necessary, as known to those skilled in the art.

As used herein, the term ‘counter ion’ refers to the ion that accompanies an ionic species to maintain electric neutrality.

As used herein, “effective amount” refers to a dose or concentration of a drug that produces a biological response.

As used herein, the term ‘half maximal inhibitory concentration (IC₅₀)’ refers to the measure of the potency of a substance in inhibiting a specific biological or biochemical function. IC₅₀ is a quantitative measure that indicates how much of a particular inhibitory substance (e.g., drug) is needed to inhibit, in vitro, a given biological process or biological component by 50%. The biological component can be an enzyme, cell, cell receptor, or microorganism. IC₅₀ values are typically expressed as molar concentration.

As used herein, the terms “treat,” “treatment,” and “treating” in the context of the administration of a therapy to a subject in need thereof refer to the reduction or inhibition of the progression and/or duration of cancer, the reduction or amelioration of the severity of cancer, and/or the amelioration of one or more symptoms thereof resulting from the administration of one or more therapies. In some embodiments, the subject is a mammalian subject. In one embodiment, the subject is a human. “Treating” or “treatment” of a disease includes preventing the disease from occurring in a subject that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease). With regard to cancer or hyperplasia, these terms simply mean that the life expectancy of an individual affected with cancer will be increased or that one or more of the symptoms of the disease will be reduced.

In specific embodiments, such terms refer to one, two or three or more results following the administration of one, two, three or more therapies: (1) a stabilization, reduction or elimination of the cancer stem cell population; (2) a stabilization, reduction or elimination in the cancer cell population; (3) a stabilization or reduction in the growth of a tumor or neoplasm; (4) an impairment in the formation of a tumor; (5) eradication, removal, or control of primary, regional and/or metastatic cancer; (6) a reduction in mortality; (7) an increase in disease-free, relapse-free, progression-free, and/or overall survival, duration, or rate; (8) an increase in the response rate, the durability of response, or number of patients who respond or are in remission; (9) a decrease in hospitalization rate, (10) a decrease in hospitalization lengths, (11) the size of the tumor is maintained and does not increase or increases by less than 10%, preferably less than 5%, preferably less than 4%, preferably less than 2%, and (12) an increase in the number of patients in remission.

In certain embodiments, such terms refer to a stabilization or reduction in the cancer stem cell population. In some embodiments, such terms refer to a stabilization or reduction in the growth of cancer cells. In some embodiments, such terms refer to stabilization or reduction in the cancer stem cell population and a reduction in the cancer cell population. In some embodiments, such terms refer to a stabilization or reduction in the growth and/or formation of a tumor. In some embodiments, such terms refer to the eradication, removal, or control of primary, regional, or metastatic cancer (e.g., the minimization or delay of the spread of cancer). In some embodiments, such terms refer to a reduction in mortality and/or an increase in a survival rate of a patient population. In further embodiments, such terms refer to an increase in the response rate, the durability of response, or a number of patients who respond or are in remission. In some embodiments, such terms refer to a decrease in hospitalization rate of a patient population and/or a decrease in hospitalization length for a patient population.

Throughout the specification and the appended claims, a given chemical formula or name shall encompass all stereo and optical isomers and racemates thereof where such isomers exist.

Unless otherwise indicated, all chiral (enantiomeric and diastereomeric) and racemic forms are within the scope of the disclosure. Many geometric isomers of C═C double bonds, C═N double bonds, ring systems, and the like can also be present in the complexes, and all such stable isomers are contemplated in the present disclosure. Cis- and trans- (or E- and Z—) geometric isomers of the complexes of the present disclosure are described and may be isolated as a mixture of isomers or as separated isomeric forms. The present complexes can be isolated in optically active or racemic forms. Optically active forms may be prepared by resolution of racemic forms or by synthesis from optically active starting materials. All processes used to prepare complexes of the present disclosure and intermediates made therein are considered to be part of the present disclosure. When enantiomeric or diastereomeric products are prepared, they may be separated by conventional methods, for example, by chromatography, fractional crystallization, or through the use of a chiral agent. Depending on the process conditions, the end products of the present disclosure are obtained either in free (neutral) or salt form. Both the free form and the salts of these end products are within the scope of the disclosure. If so desired, one form of a compound may be converted into another form. A free base or acid may be converted into a salt; a salt may be converted into a free compound or another salt; a mixture of isomeric complexes of the present disclosure may be separated into individual isomers. Complexes of the present disclosure, free form, and salts thereof, may exist in multiple tautomeric forms, in which hydrogen atoms are transposed to other parts of the molecules, and the chemical bonds between the atoms of the molecules are consequently rearranged. It should be understood that all tautomeric forms, insofar as they may exist, are included within the disclosure. Further, a given chemical formula or name shall encompass all conformers, rotamers, or conformational isomers thereof where such isomers exist. Different conformations can have different energies, can usually interconvert, and are very rarely isolatable. There are some molecules that can be isolated in several conformations. For example, atropisomers are isomers resulting from hindered rotation about single bonds where the steric strain barrier to rotation is high enough to allow for the isolation of the conformers. It should be understood that all conformers, rotamers, or conformational isomer forms, insofar as they may exist, are included within the present disclosure.

Aspects of the present disclosure are directed to a method for treating cancer using gold (I) thione complexes. The complexes are effective for decreasing an amount of cancer cells and tumor suppression. The complexes show a high degree of selectivity toward cancerous cells at low concentrations, thereby circumventing the drawbacks of the prior art, such as drug-induced toxicity.

According to an aspect of the present disclosure, a method of treating a cancer is provided. In a preferred embodiment, the cancer is cervical cancer, osteosarcoma, or colon cancer. Although the description herein refers to the use of the composition for the treatment of cervical cancer, osteosarcoma, or colon cancer, it may be understood by a person skilled in the art, that aspects of the present disclosure may be directed towards the treatment of other cancers such as, cancer of the thyroid, endocrine system, brain, breast, cervix, ovary, sarcoma, stomach, uterus medulloblastoma, colon, head and neck, liver, kidney, lung, non-small cell lung, melanoma, mesothelioma, or pancreatic cancer.

The method includes administering to a subject in need thereof a complex. In the present disclosure, the complex is of formula (I),

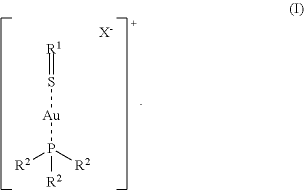

R² is a straight or branched aliphatic carbon chain including 3-10 carbon atoms, preferably 3, 4, 5, 6, 7, 8, 9, or 10 carbon atoms. In some embodiments, R² is an optionally substituted alkyl group. In some embodiments, each of the R² may be the same or different. In a preferred embodiment, each R² is a tert-butyl group.

‘X’ is a counter ion. Non-limiting examples of pharmaceutically counter-anions include halides such as fluoride, chloride, bromide, iodide; nitrate; sulfate; phosphate; amide; methanesulfonate; ethanesulfonate; p-toluenesulfonate, salicylate, malate, maleate, succinate, tartarate; citrate; acetate; perchlorate; trifluoromethane sulfonate (triflate); acetylacetonate; hexafluorophosphate; and hexafluoroacetylacetonate. In a preferred embodiment, ‘X’ is hexafluorophosphate.

R¹ is a substituted or unsubstituted cyclic amine. As used herein a cyclic amine has a carbon ring structure where at least one of the carbons is replaced by a nitrogen. Preferably 2 or 3 carbons in the ring structure are replaced with a nitrogen. In some embodiments, a ring of the cyclic amine has 5-7 atoms, where 2-3 of the atoms are nitrogen and the others are carbon. In some embodiments, the cyclic amine is saturated or unsaturated. In some embodiments, the cyclic amine is not aromatic. In a preferred embodiment, the cyclic amine is fully saturated. In a preferred embodiment, the cyclic amine has the formula of Ia,

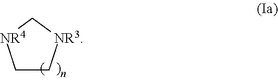

In formula (Ia) n=0, 1, or 2, to form the ring with 5-7 atoms. In some embodiments, R³ and R⁴ are each independently a hydrogen, a halogen, a phenyl, or a substituted or unsubstituted aliphatic chain having 1-10 carbon atoms, preferably 1, 2, 3, 4, 5, 6, 7, 8, 9, or 10 carbon atoms.

As referred to throughout, substituted at a nitrogen position of the cyclic amine refers to a substitution at R³ and R⁴.

In an embodiment, n=1, and R³ and R⁴ are hydrogens, and the complex has the structure of formula (II),

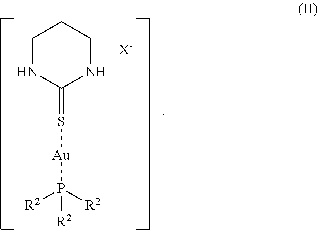

In an embodiment, n=2, and R³ and R⁴ are hydrogens, and the complex has the structure of formula (III),

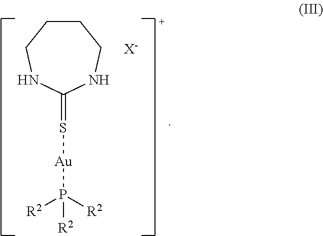

In some embodiments, n=1, and one of R³ and R⁴ is substituted with an unsubstituted aliphatic chain, including 1-10 carbon atoms at a nitrogen position of the cyclic amine. In an embodiment, the unsubstituted aliphatic chain includes 2 carbon atoms at a nitrogen position of the cyclic amine and has a structure of formula (IV),

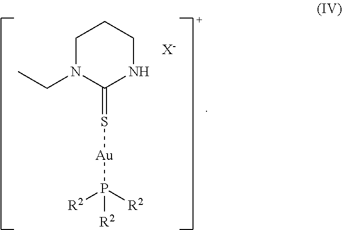

In some embodiments, n=0, and one of R³ and R⁴ is substituted with a substituted aliphatic chain, including 1-10 carbon atoms and a terminal alcohol at a nitrogen position of the cyclic amine. In an embodiment, the substituted aliphatic chain includes 2 carbon atoms and a terminal alcohol at a nitrogen position of the cyclic amine and has a structure of formula (V),

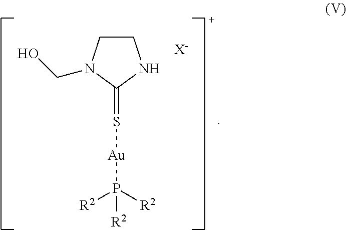

The term ‘subject’ as used herein refers to organisms to be treated by the complex of the present invention. Such organisms include animals (domesticated animal species, wild animals), and humans. In the preferred embodiment, the subject is the human. In some embodiments the subject is at least 0, 1, 2, 3, 4, 5, 10, 15, 20, 25, 30, 35, 40, 45, 50, 55, 60, 65, 70, 75, 80, 85, 90 or >90 years of age. A subject may be male or female and may have a newly diagnosed disease, disorder or condition, be currently under treatment, or suffer from a relapsing or chronic disease, disorder or condition. A subject may be someone who has lost or is losing responsiveness to another treatment, such to a cancer treatment with cisplatin or another platinum-based anticancer agent. In a preferred embodiment, the subject is resistant to or unresponsive to cisplatin treatment. A subject may have a comorbid condition such as diabetes, heart disease, hypertension (high blood pressure), hyperlipidemia (high cholesterol) and peripheral vascular disease.

A subject may have experienced one or more side-effects of another kind of treatment, such as side-effects to a cancer treatment with cisplatin or another platinum containing anticancer agent. Side effects include one or more of the following: bone marrow suppression, neurotoxicity, ototoxicity or hearing problems, nephrotoxicity or kidney problems, electrolyte disturbance, nausea, vomiting, numbness, trouble walking, allergic reactions, electrolyte problems including hypomagnesaemia, hypokalaemia and hypocalcaemia, and/or heart disease.

Those skilled in the art can determine a suitable mode for administering a gold (I) thione complex of the present disclosure based on patient status, type of disorder, disease or condition, or anatomical location of cells associated with the disorder, disease or condition. In a preferred embodiment, the administering is by oral administration, parenteral administration, topical application, trans-dermally or nasally. The term “parenteral”, as used herein, includes subcutaneous, intravenous, intramuscular, and intrasternal injection, or infusion techniques. For therapeutic purposes, formulations for parenteral administration can be in the form of aqueous or non-aqueous isotonic sterile injection solutions or suspensions. Administration may be targeted to a specific site or anatomical compartment containing cancer cells including into a solid cancer or into the vasculature or other compartments for a non-solid cancer.

In some embodiments, a gold complex of the present disclosure may be administered as the only active agent. In other embodiments, more than one type of gold complex of the present disclosure may be administered.

In certain embodiments, the complex of the present disclosure or pharmaceutical composition thereof may be used in combination with one or more other antineoplastic or chemotherapeutic agents. A non-limiting list of examples of chemotherapeutic agents are aflibercept, asparaginase, bleomycin, busulfan, carmustine, chlorambucil, cladribine, cyclophosphamide, cytarabine, dacarbazine, daunorubicin, doxorubicin, etoposide, fludarabine, gemcitabine, hydroxyurea, idarubicin, ifosfamide, irinotecan, lomustine, mechlorethamine, melphalan, mercaptopurine, methotrexate, mitomycin, mitoxantrone, pentostatin, procarbazine, 6-thioguanine, topotecan, vinblastine, vincristine, retinoic acid, oxaliplatin, cisplatin, carboplatin, 5-FU (5-fluorouracil), teniposide, amasacrine, docetaxel, paclitaxel, vinorelbine, bortezomib, clofarabine, capecitabine, actinomycin D, epirubicine, vindesine, methotrexate, tioguanine (6-thioguanine), tipifarnib. Examples for antineoplastic agents which are protein kinase inhibitors include imatinib, erlotinib, sorafenib, sunitinib, dasatinib, nilotinib, lapatinib, gefitinib, temsirolimus, everolimus, rapamycine, bosutinib, pzopanib, axitinib, neratinib, vatalanib, pazopanib, midostaurin and enzastaurin. Examples for antineoplastic agents which are antibodies comprise trastuzumab, cetuximab, panitumumab, rituximab, bevacizumab, mapatumumab, conatumumab, lexatumumab and the like.

Other embodiments of the invention include the gold complexes, per se, as well as compositions, such as those containing one or more gold complexes as disclosed herein and encompassed by Formula (I) in combination with a pharmaceutically acceptable excipient or carrier or in combination with another active agent. Typically, a pharmaceutical composition containing a gold(I) complex is sterile, isotonic, and otherwise suitable for administration to a patient.

As used herein, the term “composition” refers to a mixture of the active ingredient with other chemical components, such as pharmaceutically acceptable carriers and excipients. The composition may be manufactured using any of a variety of processes, including, without limitation, conventional mixing, dissolving, granulating, levigating, emulsifying, encapsulating, entrapping, and lyophilizing. The pharmaceutical composition can take any of a variety of forms including, without limitation, a sterile solution, suspension, emulsion, lyophilisate, tablet, pill, pellet, capsule, powder, syrup, elixir or any other dosage form suitable for administration.

As used herein, the term “active ingredient” refers to an ingredient in the composition that is biologically active, for example, the gold(I) complex of Formula (I), a salt thereof, a solvate thereof, a tautomer thereof, and a stereoisomer thereof.

As used herein, the phrase “pharmaceutically acceptable carrier or excipient” refers to a pharmaceutically acceptable material, composition or vehicle such as a liquid or solid filler, diluent, binder, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient.

Exemplary materials which can serve as pharmaceutically acceptable carriers include, but are not limited to: (1) sugars, such as lactose, glucose and sucrose; (2) starches, such as corn starch and potato starch; (3) cellulose, and its derivatives, such as sodium carboxy methyl cellulose, ethyl cellulose and cellulose acetate; (4) powdered tragancanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) other non-toxic compatible substances employed in pharmaceutical formulations and mixtures thereof. Non-limiting examples of specific uses of pharmaceutically acceptable carriers can be found in, e.g. “Pharmaceutical Dosage Forms and Drug Delivery Systems” (Howard C. Ansel et al., eds., Lippincott Williams & Wilkins Publishers, 11th edition, 2017; ISBN-13: 978-1496347282); “Remington: The Science and Practice of Pharmacy” (Alfonso R. Gennaro ed., Lippincott, Williams & Wilkins, 21th edition 2005; 0-7817-4673-6); “Goodman & Gilman's The Pharmacological Basis of Therapeutics” Joel G. Hardman et al., eds., McGraw-Hill Professional, 13th edition. 2017, 1259584739); and “Handbook of Pharmaceutical Excipients” (Raymond C. Rowe et al., APhA Publications, 5th edition, 2005; 1582120587), each incorporated herein by reference in their entirety.

In another embodiment, wetting agents, emulsifiers and lubricants, such as sodium lauryl sulfate and magnesium stearate, as well as coloring agents, release agents, coating agents, sweetening, flavoring and perfuming agents, preservatives and antioxidants may also be present in the compositions described herein. Exemplary pharmaceutically acceptable antioxidants include, but are not limited to: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In another embodiment, the pharmaceutically acceptable carrier or excipient is a binder. As used herein, “binders” refers to materials that hold the ingredients in a tablet together. Binders ensure that tablets and granules can be formed with the required mechanical strength, and give volume to low active dose tablets. Exemplary pharmaceutically acceptable binders include, but are not limited to: (1) saccharides and their derivatives, such as sucrose, lactose, starches, cellulose or modified cellulose such as microcrystalline cellulose, carboxy methyl cellulose, and cellulose ethers such as hydroxypropyl cellulose (HPC), and sugar alcohols such as xylitol, sorbitol or maltitol; (2) proteins such as gelatin; and (3) synthetic polymers including polyvinylpyrrolidone (PVP) and polyethylene glycol (PEG).

Binders can be classified according to their application. Solution binders are dissolved in a solvent (i.e. water or alcohol in wet granulation processes). Exemplary solution binders include, but are not limited to, gelatin, cellulose, cellulose derivatives, polyvinylpyrrolidone, starch, sucrose and polyethylene glycol. Dry binders are added to the powder blend, either after a wet granulation step, or as part of a direct powder compression (DC) formula. Exemplary dry binders include, but are not limited to, cellulose, methyl cellulose, polyvinylpyrrolidone and polyethylene glycol. In terms of the present disclosure, the pharmaceutically acceptable carrier or excipient may be a solution binder, a dry binder or mixtures thereof.

In some embodiments, the pharmaceutically acceptable carrier and/or excipient used herein may be an organic solvent, an inorganic salt, a surfactant, and/or a polymer.

Exemplary inorganic salts include, without limitation, calcium carbonate, calcium phosphate, disodium hydrogen phosphate, potassium hydrogen phosphate, sodium chloride, zinc oxide, zinc sulfate, and magnesium trisilicate.

Surfactants that may be present in the compositions of the present disclosure include zwitterionic (amphoteric) surfactants, e.g., phosphatidylcholine, and 3-[(3-cholamidopropyl)dimethylammonio]-1-propanesulfonate (CHAPS), anionic surfactants, e.g., sodium lauryl sulfate, sodium octane sulfonate, sodium decane sulfonate, and sodium dodecane sulfonate, non-ionic surfactants, e.g., sorbitan monolaurate, sorbitan monopalmitate, sorbitan trioleate, polysorbates such as polysorbate 20 (Tween 20), polysorbate 60 (Tween 60), and polysorbate 80 (Tween 80), cationic surfactants, e.g., decyltrimethyl ammonium bromide, dodecyltrimethylammonium bromide, tetradecyltrimethylammonium bromide, tetradecyltrimethyl-ammonium chloride, and dodecylammonium chloride, and combinations thereof.

Exemplary polymers include, without limitation, polylactides, polyglycolides, polycaprolactones, polyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, poly(malic acid), poly(amino acids), polyvinylpyrrolidone, polyethylene glycol, polyhydroxycellulose, chitin, chitosan, and copolymers, terpolymers, or combinations or mixtures thereof.

The composition may have <0.01, 0.01, 0.02, 0.05, 0.1, 0.2, 0.5, 1, 2, 5, 10, 20, 50, 100 or >100 μM of the gold(I) complex of formula (I) relative to the total volume of the composition.

A composition may contain <0.01, 0.01, 0.02, 0.05, 0.1, 0.2, 0.5, 1, 2, 5, 10, 20, 50 or >50 wt % of a gold(I) complex of formula (I) based on a total weight of the composition or any intermediately value within this range.

The gold(I) complexes of the invention, such as gold complex (1), may be used to modulate or upregulate the expression of caspaces such as Caspase 3 and 9, and to induce apoptosis in target cells.

Depending on the route of administration e.g. oral, parental, or topical, the composition may be in the form of solid dosage form such as tablets, caplets, capsules, powders, and granules, semi-solid dosage form such as ointments, creams, lotions, gels, pastes, and suppositories, liquid dosage forms such as solutions, and dispersions, inhalation dosage form such as aerosols, and spray, or transdermal dosage form such as patches.

Solid dosage forms for oral administration may include capsules, tablets, pills, powders, and granules. In such solid dosage forms, the active ingredients are ordinarily combined with one or more adjuvants appropriate to the indicated route of administration. If administered per os, the active ingredients can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled release formulation as can be provided in a dispersion of active compound in hydroxypropylmethyl cellulose. In the case of capsules, tablets, and pills, the dosage forms can also include buffering agents such as sodium citrate, magnesium or calcium carbonate or bicarbonate. Tablets and pills can additionally be prepared with enteric coatings.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

These solutions and suspensions can be prepared from sterile powders or granules having one or more of the pharmaceutically acceptable carriers or excipients mentioned for use in the formulations for oral administration. The active ingredients can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Injectable preparations, for example, sterile injectable aqueous or oleaginous suspensions, can be formulated according to the known art using suitable dispersing or wetting agents and suspending agents. The sterile injectable preparation may also be a sterile injectable solution or suspension in a pharmaceutically acceptable diluent or solvent. Among the pharmaceutically acceptable diluents and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and surfactants such as those discussed above are also useful.

To reduce viability or proliferation of cancer cells or abnormally proliferating cells, a subject may be treated with a concentration of a complex described herein ranging from 0.01, 0.1, 1, 5, 10, 20, 30, 40, 50, 60, 70, 80, 90 or 100 μM or with an amount that contacts the cells in the subject to a concentration of 0.01, 0.1, 1, 5, 10, 20, 30, 40, 50, 60, 70, 80, 90 or 100 μM of the gold(I) complex. The complex has an IC₅₀ of 1-15 μM, preferably 2-14 μM, 4-12 μM, 6-10 μM, or about 8 μM against colon cancer cells in the subject. The complex has an IC₅₀ of 3-40 PM, preferably 5-35 μM, 10-30 μM, 15-25 μM, or about 20 μM against cervical cancer cells in the subject. The complex has an IC₅₀ of 0.1-15 μM, preferably 1-12 μM, 4-10 μM, or 6-8 μM against osteosarcoma cancer cells in the subject.

In an implementation of the present disclosure, the complex may be orally administered based on the body weight of the subject. Particularly, an effective amount of the complex is determined based on the body weight of the subject. In some embodiments, the administering is 1-20 milligrams (mg) of the complex per kilogram (kg) of the subject, preferably 2-18 mg/kg, 4-16 mg/kg, 6-14 mg/kg, 8-12 mg/kg, or about 10 mg/kg. Further, the effective amount of the complex may be determined based on various factors such as age of the subject, medical condition of the subject, body weight of the subject, etc.

Following administration, the amount of colon cancer cells in the subject is reduced by at least 50%, preferably 60%, and more preferably 80%. Following administration, the amount of cervical cancer cells in the subject is reduced by at least 40%, preferably 50%, or 60%. Moreover, following administration, the amount of osteosarcoma cancer cells in the subject is reduced by at least 60%, preferably 70%, more preferably 80%. In a preferred embodiment, following the administering of the complex of formula (II) an amount of colon cancer cells or osteosarcoma cancer cells in the subject are reduced by at least 80%, preferably 85%, 90%, or 95%. In some embodiments, the complex is 5-30, preferably 10-25, or 15-20 times more effective at reducing the number of cancer cells in the subject than cisplatin.

In some embodiments, the interval of time between the administration of gold complexes or pharmaceutical composition thereof and the administration of one or more additional therapies may be about 1-5 minutes, 1-30 minutes, 30 minutes to 60 minutes, 1 hour, 1-2 hours, 2-6 hours, 2-12 hours, 12-24 hours, 1-2 days, 2 days, 3 days, 4 days, 5 days, 6 days, 7 days, 1 week, 2 weeks, 3 weeks, 4 weeks, 5 weeks, 6 weeks, 7 weeks, 8 weeks, 9 weeks, 10 weeks, 15 weeks, 20 weeks, 26 weeks, 52 weeks, 11-15 weeks, 15-20 weeks, 20-30 weeks, 30-40 weeks, 40-50 weeks, 1 month, 2 months, 3 months, 4 months 5 months, 6 months, 7 months, 8 months, 9 months, 10 months, 11 months, 12 months, 1 year, 2 years, or any period of time in between. In some embodiments, if the health condition of the subject is critical, the complex may be administered daily. In some embodiments, the daily administration of the complex may depend on various factors such as the degree of progression of obesity, time of onset, age, health condition, and complications of the subject to be administered. In some embodiments, the complex of the present disclosure may be generally administered once or several times a day.

The methods for treating cancer and other proliferative disorders described herein inhibit, remove, eradicate, reduce, regress, diminish, arrest, or stabilize a cancerous tumor, including at least one of the tumor growth, tumor cell viability, tumor cell division and proliferation, tumor metabolism, blood flow to the tumor and metastasis of the tumor. In some embodiments, after treatment with one or more complexes or a pharmaceutical composition thereof, the size of a tumor, whether by volume, weight, or diameter, is reduced by at least 5%, 10%, 15%, 20%, 25%, 30%, 40%, 50%, 60%, 70%, 75%, 80%, 85%, 90%, 95%, 99% or 100%, relative to the tumor size before treatment. In other embodiments, after treatment with one or more gold complexes of pharmaceutical composition thereof, the size of a tumor may not reduce but is maintained the same as the tumor size before treatment. Methods of assessing tumor size include but are not limited to CT scan, MRI, DCE-MRI, and PET Scan.

### EXAMPLES

The following examples describe and demonstrate exemplary embodiments of the method described herein. The examples are provided solely for the purpose of illustration and are not to be construed as limitations of the present disclosure, as many variations thereof are possible without departing from the spirit and scope of the present disclosure.

### Example 1: Materials and Instrumentation

All solvents were of analytical grade and were used without further purification. Ethanol, diethyl ether, dichloromethane, acetone, were purchased from Fluka AG (St. Gallen, Switzerland). Gold(I) precursor, chlorido(tri-tert-butylphosphine)gold(I) was purchased from Strem Chemicals Inc. (Newburyport, Massachusetts. United States). AgPF₆ was purchased from Sigma-Aldrich, United States. 1,3-diazinane-2-thione (Diaz), N-hydroxyethyl, 1,3-imidazolidine-2-thione (2-EtOH-Imt), 1,3-diazepine-2-thione (Diap) and N-ethyl-1,3-diazinane-2-thione (N-Et-Diaz) were prepared.

Elemental analyses were performed on Perkin Elmer Series 11(CHNS/O), Analyzer 2400 (manufactured by Perkin Elmer, 940 Winter St, Waltham, Massachusetts, 02451, United States). Solid state Fourier transform infrared (FT-IR) spectra were recorded on a PerkinElmer FT-IR 180 spectrophotometer (manufactured by Perkin Elmer, 940 Winter St, Waltham, Massachusetts, 02451, United States) using KBr pellets over the range 4000-400-centimeter inverse (cm⁻¹) at resolution 4.00 cm⁻¹. ¹H (500.01 megahertz (MHz)), ¹³C (125.65 MHz), and ³¹P (200.0 MHz) nuclear magnetic resonance (NMR) spectra were recorded on a LAMBDA Jeol 500.01 MHz NMR spectrophotometer (manufactured by JEOL, 11 Dearborn Road Peabody, MA 01960, USA).

Spectral conditions for ¹³C NMR were: 32 k data points, 0.967 second (s) acquisition time, 1.00 s pulse delay, and 450 pulse angle. The chemical shifts are obtained with respect to tetramethylsilane (TMS) as an internal (¹H and ¹³C) and H₃PO₄ as an external (³¹P) at 0.0 parts per million (ppm).

### Example 2: Synthesis of Gold(I) Complexes (1-4)

AgPF₆ (0.127 g, 0.500 millimole (mmol)) was dissolved in 7.0 milliliters (mL) of ethanol and added to a solution of (t-Bu)₃PAuCl (0.217 g, 0.500 mmol) in acetone (10.0 mL) to obtain a mixture. The mixture was stirred at ambient temperature for 30 minutes and filtered off. Thione ligand (0.50 mmol) was added to the filtrate; the mixture was stirred for 2 hours, filtered out, and stored in an undisturbed area. After four to six days, the products were obtained. Complexes 1-4 were separated as colorless, bright yellowish-white, and yellow solids. Suitable crystals were selected for the single crystal diffraction analysis.

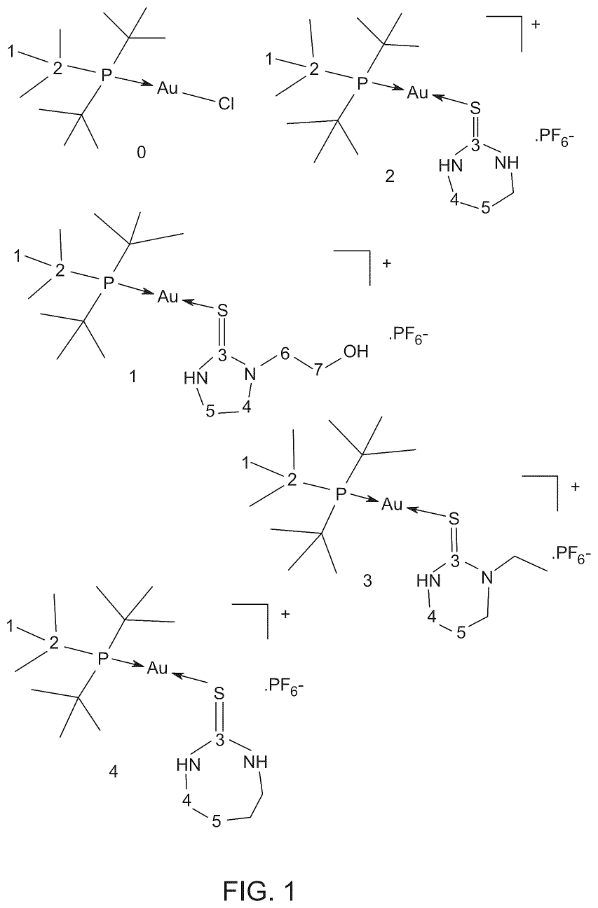

A complex labeled as Complex 0 has a chloride ligand for comparative purposes. For Complex 1 the differing ligand is N-hydroxyethyl-1,3-imidazolidine-2-thione (2-EtOH-Imt). For Complex 2 the differing ligand is diazinane-2-thione (Diaz). For Complex 3 the differing ligand is N-ethyl-1,3-diazinane-2-thione (Et-Diaz). For Complex 4 the differing ligand is 1,3-diazepine-2-thione (Diap). The complexes 0-4 are depicted in FIG. 1.

[Au{P(t-Bu)₃}(SN₂C₅H₉—OH)]PF₆ (1). Yield 0.213 g (62%). Anal. calc. for C₁₇H₃₇AuN₂OP₂SF₆ (690.46 g/mol): C, 29.57; H, 5.40; N, 4.05; S, 4.64. Found: C, 30.39; H, 4.52; N, 4.13; S, 4.55.

[Au{P(t-Bu)₃}(SN₂C₃H₆)]PF₆ (2). Yield 0.268 g (81%). Anal. calc. for C₁₆H₃₅AuN₂P₂SF₆ (660.43 g/mol): C, 29.09; H, 5.34; N, 4.24; S 4.85. Found: C, 31.26; H, 3.99; N, 3.01; S, 3.03.

[Au{P(t-Bu)₃}(SN₂C₅H₁₀]PF₆ (3). Yield 0.213 g (62%). Anal. Calc. for C₁₈H₃₉AuN₂P₂SF₆ (688.49 g/mol): C, 31.40; H, 5.70; N, 4.06; S, 4.65. Found: C, 31.52; H, 4.24; N, 4.57; S, 4.97.

[Au{P(t-Bu)₃}(SN₂C₅H₁₀]PF₆ (4). Yield 0.192 g (57%). Anal. Calc. for C₁₇H₃₇AuN₂P₂SF₆ (674.46 g/mol): C, 30.27; H, 5.52; N, 4.15; S, 4.75. Found: C, 30.91; H, 4.16; N, 4.70; S, 4.94.

### Example 3: X-Ray Diffraction Measurements

The X-ray data of 3 and 4 were collected at 173K (−100° C.) on a STOE image plate diffraction system (IPSD 2) connected with a two-circle goniometer and using MoKα graphite monochromator (λ=0.71073 angstrom (Å)). Diffraction data were collected using a detector image plate (34 diameters) and sealed X-ray tube diffraction source 12×0.4 mm long fine focus. The structure was solved by the SHELXS-2014 program. The refinement and further calculations were carried out with SHELXL-2014. N—H H atoms were located in a Difference Fourier map and refined with a distance restraint of N—H=0.88(2) A and H . . . H=1.40(2) Å. C-bound H-atoms were included in calculated positions and treated as riding atoms: C—H=0.95-1.0 Å with Uᵢₛₒ(H)=1.5 U_{eq}(C) for methyl H atoms and =1.2 U_{eq}(C) for other H-atoms. Non-H atoms were refined anisotropically using weighted full-matrix least squares on F2. A semi-empirical absorption correction was applied using the MUL scan ABS routine in PLATON. The F atoms of the PF₆⁻ anion is disordered. The best solution was found by distributing the electron density over 11 positions, refined with various fixed occupancy ratios to give a total of six F atoms. A summary of crystal data and refinement details for complexes 3 and 4 are shown in Table 1.

TABLE 1
Summary of crystal data and details of the structure
refinement for the complexes (3 and 4).

| Complex | 3 | 4 |
|---|---|---|
| Crystal data | | |
| Empirical formula | C₁₈H₃₉AuF₆N₂P₂S | C₁₇H₃₇AuF₆N₂P₂S |
| Formula weight | 688.49 g/mol | 674.46 g/ mol |
| Crystal symmetry | Monoclinic | Monoclinic |
| Space group | P 21/c | P 21/c |
| Crystal color | Colorless | Colorless |
| Crystal description | Block | — |
| Crystal size / mm | 0.40 × 0.33 × 0.19 | — |
| Wavelength/Å | 0.71073 | 0.71073 |
| Temperature/K | 233(2) | 293(2) |
| Cell length a (Å) | 24.9014(16) | 16.053(4) |
| Cell length b (Å) | 13.0453(5) | 11.756(3) |
| Cell length c (Å) | 16.0089(9) | 26.392(4) |
| Cell angles α (°) | 90 | 90 |
| Cell angles β (°) | 90 | 90 |
| Cell angles γ (°) | 90.764 (5) | 90.97 |
| Dₓ | 1.686 Mg m⁻³ | 1.686 Mg m⁻³ |
| M | 5.92 mm⁻¹ | 6.16 mm⁻¹ |
| Radiation type | Mo Kα | Mo Kα |
| Cell volume (Å³) | 5197.9 (5) | 4979.96(3) |
| Z | 8 | — |
| Data collection | | |
| Diffractometer | STOE IPDS 2 | STOE IPDS 2 |
| Absorption correction | Multi-scan (MULABS in | — |
| | PLATON; 2009 | |
| Radiation source | Fine-focus sealed tube | Plane graphite |
| Refinement | | |
| R[F² > 2σ(F²)], wR(F²), S | 0.36, 0.087, 0.96 | — |
| Δρₘₐₓ | 1.14 e Å⁻³ | 1.01 e Å⁻³ |
| Δρₘᵢₙ | −1.38 e Å⁻³ | −1.50 e Å⁻³ |
| H-atom treatment | Treated by a mixture of | Treated by a mixture of |
| | independent and constrained | independent and constrained |
| | refinement. | refinement. |

### Example 4: Measurement of Anticancer Activity Method

The complexes, [Au{(t-Bu)₃P}Cl] and (1-4) were tested for their in vitro cytotoxic effects against human cell lines; HCT15 (colon cancer), MG-63 (Human osteosarcoma (human bone cancer)), and HeLa (human cervical cancer). The cells were seeded at the concentration of 3×10³ cells/well in 100 microliters (L) of Dulbecco's modified eagle medium (DMEM) containing 10% fetal bovine serum (FBS) in a 96-well tissue culture plate and incubated for 72 hours (h) at 37° C., 5% CO₂ and 90% relative humidity in a CO₂ incubator. After that, 100 μL of 25.0, 12.5, 6.25, and 3.125 μM solutions of cisplatin and the gold(I) complexes 0-4 prepared in DMEM were added to the cells, and the cultures were incubated for 72 h. Then, the medium in wells was cast off, and 100 μL of DMEM containing MTT (3-(4,5-dimethylthiazol-2-yl)-2,5-diphenyltetrazolium bromide) (0.5 mg/ml) was added to the wells, with subsequent incubation in the CO₂ incubator at 37° C. in the dark for 4 h. After incubation, purple-colored formazan produced by the cells appeared as dark crystals in the bottom of the wells. The culture medium was carefully removed from each well to prevent monolayer disruption, and 100 μL of dimethylsulfoxide (DMSO) was added to each well. The solution in the wells was thoroughly mixed to dissolve the formazan crystals, which produced a purple solution. The absorbance of the 96 well-plates was measured at 570 nm with LabSystems Multiskan EX-ELISA reader (manufactured by Thermo Fischer Scientific, 168 Third Avenue Waltham, MA 02451 USA) against a reagent blank. The experimental results are calculated as the micromolar concentration of 50% cell growth inhibition (IC₅₀) of each drug. The MTT assay was carried out in three independent experiments for each analysis.

### Example 5: Semi-Quantitative Reverse Transcription-Polymerase Chain Reaction (RT-PCR) Method

The underlying molecular mechanism for the anti-cancer activity of the gold complexes was determined by using the central dogma theory. The gene expression of caspase family proteins, i.e., caspase 3 (CASP3) and caspase 9 (CASP9) was examined by constructing the first transcript (cDNA) from their respective mRNA by reverse transcription. Further, the cDNA was amplified and electrophoresed on 2% agarose gel. β-actin (ACTB) was used as an internal control. The primer pairs for the desired genes were custom synthesized from Bioserve Biotechnologies (Hyderabad, India) (Table 2).

TABLE 2
Primer's sequence used for semi-quantitative RT-PCR

| Sr. | | | | PCR |
|---|---|---|---|---|
| no. | mRNA | Primers | Sequence (5′à3′) | Product |
| 1 | CASP9 | Forward | ATGATCGAGGACATCCAGCG | 266 bp |
| | | Reverse | CTGGGTGTTTCCGGTCTGAG | |
| 2 | CASP3 | Forward | CTCGGTCTGGTACAGATGTCG | 263 bp |
| | | Reverse | ACTTCTACAACGATCCCCTCTG | |
| 3 | ACTB | Forward | TCACCCACACTGTGCCCATCTACGA | 295 bp |
| | | Reverse | CAGCGGAACCGCTCATTGCCAATGG | |

### Example 6: In Vivo Animal Studies Methods

The animal experiments were carried out per guidelines approved by the King Saud University Research Ethics Committee (KSU-SE-20-62). 6- to 8-week-old male nude mice were provided by the university's experimental surgery and animal laboratory. The 6- to 8-week-old male nude mice were housed in clean cages during the experiment (25° C. with a 12-hr dark/light cycle). Human colorectal cell lines (HCT-116) were prepared at 1×10⁶ cells in 50 μL of DMEM medium mixed with 50 μL of Matrigel basement membrane matrix (BD Biosciences, San Jose, CA, USA) and then were injected into subcutaneous tissue. Four days after inoculation, complex 2 (8 milligrams per kilogram (mg/kg) in 100 μL 5% DMSO and Phosphate buffered saline (PBS)) was administered to mice weekly by intraperitoneal (i.p.) injection for three weeks. The tumor volume was measured using a two-dimensional mathematical model (TV)=((L*W\hat{ }2))/2. Finally, the mice were sacrificed, and the tumor and blood were collected for further investigation.

### Example 7: Spectroscopic Characterization

Complex 0, chlorido(tri-tert-butylphosphine)gold(I), was treated with AgPF₆ in ethanol to remove chloride ions as AgCl. Then, the complexes (1-4) were obtained by adding one equivalent of thione ligand to the filtrate of complex 0. The obtained products (1-4) have a composition of [Au(tri-tert-butylphosphane)(thione)]PF₆ as shown in (FIG. 1) as indicated by ¹H, ¹³C, ³¹P NMR, mid-FTIR spectroscopy, elemental analyses, and single crystal XRD analysis. The complexes (1-4) are mononuclear molecules possessing linear geometry at the gold center.

The mid-FTIR spectroscopy data of free ligands and their complexes are summarized in Table 3. In FT-IR spectra of complexes 1-4, the bands in the range of (830-870 cm⁻¹) were assigned to v(C═S) stretching vibration, which is shifted with respect to their positions in free ligands (1001-1191 cm⁻¹). This shift indicates a decrease in the double bond character and weakening of the C═S bond upon coordination. In complexes 1-4, the strong v(C—N═S) stretching vibration band was assigned in the range of (1443-1480) cm⁻¹ due to increasing double character in the C—N bond. The N—H and O—H stretching shifts to a higher frequency region (3402, 3600, 3379, 3409 vs. 3224, 3263, 3266, 3290 cm⁻¹ for free ligands).

TABLE 3
Mid-FTIR frequencies (cm⁻¹) of free ligand and (tri-
tert-butylphosphino) gold(I) thione complexes (1-4).

| | Ligand | | | | | | |
|---|---|---|---|---|---|---|---|
| | | | | | | Stretch | Stretch |
| Complex | Stretch NH/OH | | | | | N—C | C═S |
| 2-EtOH-Imt | 3290 | 2979 | 1379 | 2925 | 1358 | 1466 | 1065 |
| Diaz | 3266 | — | — | 2924 | 1360 | 1488 | 1066 |
| Et-Diaz | 3263 | — | — | 2935 | 1315 | 1448 | 1191 |
| Diap | 3224 | — | — | 2922 | 1347 | 1445 | 1001 |
| 1 | 3600 (OH) | 2999 | 1368 | 2953, 2895 | 1328 | 1476 | 830 |
| 2 | 3402 (NH) | 3000 | 1370 | 2954, 2870 | 1319 | 1480 | 875 |
| 3 | 3409 (NH) | 2952 | 1375 | 2904, 2870 | 1258 | 1443 | 852 |
| 4 | 3379 (NH) | 3005 | 1370 | 2952, 2861 | 1223 | 1480 | 846 |

The ¹H NMR chemical shifts of free thione ligands and their complexes are given in Table 4. For example, ¹H NMR data for complex 2 display a doublet at 1.48 ppm, triplet at 3.23 ppm, and triplet at 1.77 ppm. While complex 0 shows a doublet at 1.51 ppm. Also, the N—H signals of thiones in complexes shifted downfield compared to their free position values. The de-shielding is related to an increase in π character of the C—N bond due to the flow of electron density from nitrogen toward sulfur upon coordination.

TABLE 4
¹H NMR chemical shifts (ppm) for free ligands and (tri-tert-
butylphosphino) gold(I) thione complexes (0-4) in DMSO.

| | Ligand | | | | | | | |
|---|---|---|---|---|---|---|---|---|
| complex | 1H | 4H | 5H | 6H | 7H | 8H | NH | OH |
| 0 | 1.51 d | — | — | — | — | — | — | — |
| 2-EtOH-Imt | — | 3.48 t | 3.67 t | 3.52 t | 3.37 t | — | 7.93 | 4.75 |
| 1 | 1.48 d | 3.52 t | 3.89 t | 3.62 t | 3.45 t | — | 8.95 | 4.98 |
| Diaz | — | 3.07 t | 1.70 m | — | — | — | 7.76 | — |
| 2 | 1.48 d | 3.23 t | 1.77 m | — | — | — | 8.93 | — |
| Et-Diaz | — | 3.05 t | 1.80 m | 3.26 t | 3.71 q | 1.04 t | 7.71 | — |
| 3 | 1.47 d | 3.21 t | 1.86 m | 3.44 t | 3.77 q | 1.17 t | 8.97 | — |
| Diap | — | — | — | — | — | — | — | — |
| 4 | 1.48 d | 3.48 t | 1.85 m | — | — | — | 7.20 | — |

The chemical shifts of ¹³C and ³¹P NMR for free ligand and Au(I) complexes 0-4 are shown in Table 5. ¹³C NMR spectra support the proposed structure of the synthesized complexes. The C═S resonance was observed in free ligands at 182.4, 173.9, 175.9, and 183.9 ppm, respectively. It shifted to upfield by 4-7 ppm in all complexes at 175.2, 166.8, 167.4, and 179.4 ppm, respectively. The shift is consistent with the back donation from gold ion to the π*C═S. The C4 (C—N) resonance was shifted downfield in all complexes relative to the free ligand, due to the increase in the sp² character of nitrogen upon coordination.

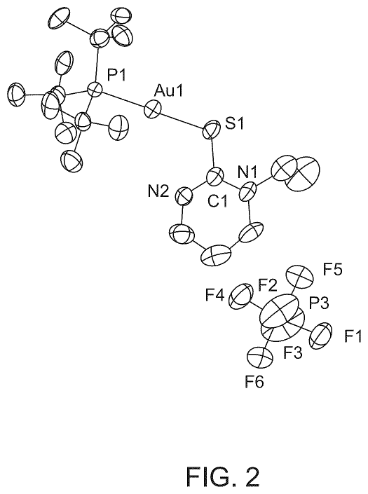

³¹P NMR spectra agree with ¹H and ¹³C NMR results. The ³¹P resonance appears at 90.0 ppm for complex 0, thus being shifted downfield upon coordination in all complexes as a doublet at 97.0, 92.5, 99.2, and 92.2 ppm, respectively. A linear correlation between ³¹P NMR chemical shift and ¹³C═S NMR chemical shifts due to the increased ring constraint of complexes (1-4) is shown in FIG. 2. The PF₆ counter ion appeared at −141.2, −146.0, −141.2, and 146.1 ppm, septet respectively, for complexes (1-4).

TABLE 5
¹³C and ³¹P NMR chemical shifts (ppm) for free ligands and (tri-
tert-butylphosphino)gold(I) thione complexes (1-4) in DMSO.

| | Ligand | | | | | | | | | |
|---|---|---|---|---|---|---|---|---|---|---|
| | | | | | | | | | ³¹P | |
| Complex | C1 | C2 | C═S | C4 | C5 | C6 | C7 | C8 | P | PF₆⁻ |
| 0 | 32.2 | 39.5 | — | — | — | — | — | — | 90.0 | — |
| 2-EtOHImt | — | — | 182.4 | 40.9 | 48.5 | 49.1 | 58.8 | — | — | |
| 1 | 32.2 | 40.4 | 175.2 | 42.5 | 49.8 | 50.3 | 58.5 | — | 97.0 d | −141.2 |
| | | | | | | | | | | Septet |
| Diaz | — | — | 173.9 | 40.1 | 18.6 | — | — | — | — | |
| 2 | 31.8 | 40.9 | 166.8 | 41.2 | 18.4 | — | — | — | 92.5 d | −146.0 |
| | | | | | | | | | | septet |
| EtDiaz | — | — | 175.9 | 40.2 | 20.9 | 45.0 | 47.6 | 12.1 | — | |
| 3 | 32.2 | 40.4 | 167.4 | 40.7 | 20.2 | 46.6 | 49.4 | 12.9 | 99.2 d | −141.2 |
| | | | | | | | | | | septet |
| Diap | — | — | 183.9 | 45.9 | 26.9 | — | — | — | — | |
| 4 | 32.3 | 39.7 | 179.4 | 46.5 | 26.1 | — | — | — | 92.2 d | −146.1 |
| | | | | | | | | | | septet |

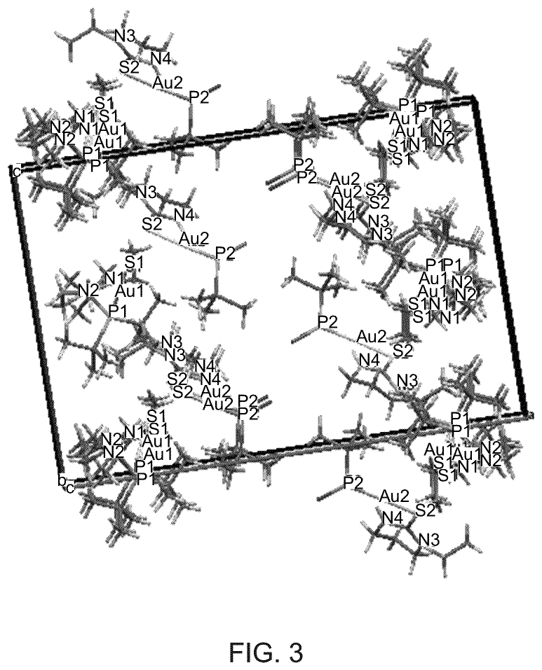

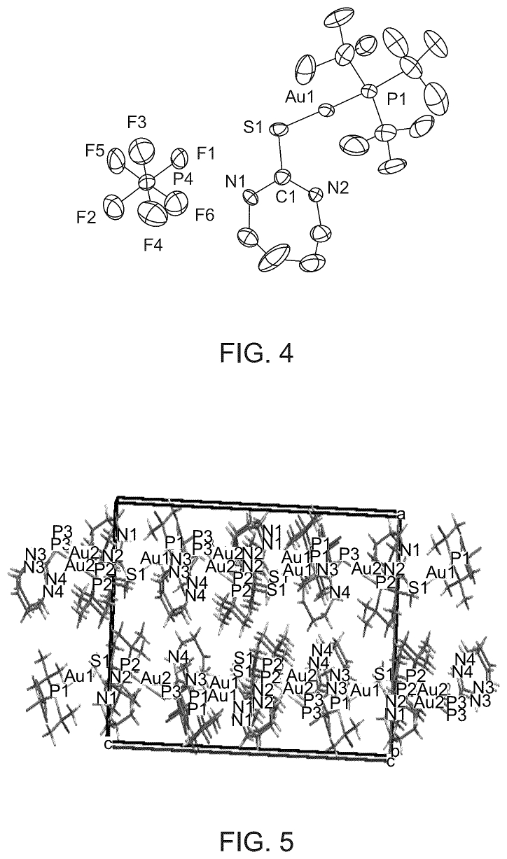

The molecular structure and crystal packing of the complexes [Au{P(t-Bu)₃}(SN₂C₅H₁₀]PF₆ (3) and [Au{P(t-Bu)₃}(SN₂C₅H₁₀]PF₆ (4) are depicted in FIGS. 2-5 respectively. The selected bond lengths and angles for complexes 3 and 4 are shown in Table 6. The coordination geometry around gold ion is close to linearity, with (P1-Au—S1) 178.1 (7°) and 178.0 (5°), respectively. These values reflect that the geometry at gold is somewhat distorted linear. Complexes 3 and 4 are isostructural and possess similar bond parameters. The Au1-P1 and Au1-S1 bond lengths are 2.23 (2); 2.32 (1); 2.27 (2); 2.29 (1) Å, respectively. No evidence of aurophilic interactions was found in the crystal structures of 3 and 4, which may be due to the steric bulk of the tri-(tri-tert-butylphosphine and thiones ligands attached to gold(J) ion.

TABLE 6
Selected bond lengths and bond angles for complexes 3 and 4.

| | |
|---|---|
| Bond Length (Å) | Bond Angles (°) |

Complex 3

| | | | |
|---|---|---|---|
| Au1—S1 | 2.32 (1) | S1—Au1—P1 | 178.0 (5) |
| Au1—P1 | 2.29 (2) | Au1—S1—C1 | 103.8 (2) |
| S1—C1 | 1.74 (5) | S1—C1—N1 | 121.0 (4) |
| N1—C1 | 1.31 (7) | S1—C1—N2 | 120.3 (4) |
| N2—C1 | 1.32 (9) | N1—C1—N2 | 118.6 (5) |

Complex 4

| | | | |
|---|---|---|---|
| Au1—S1 | 2.32 (2) | S1—Au1—P1 | 178.13 (7) |
| Au1—P1 | 2.27 (2) | Au1—S1—C1 | 100.8 (2) |
| S1—C1 | 1.74 (7) | S1—C1—N1 | 119.6 (5) |
| N1—C1 | 1.32 (8) | S1—C1—N2 | 117.1 (5) |
| N2—C1 | 1.31 (3) | N1—C1—N2 | 123.3 (6) |

### Example 8: In-Vitro Cytotoxicity of Gold(J) Complexes

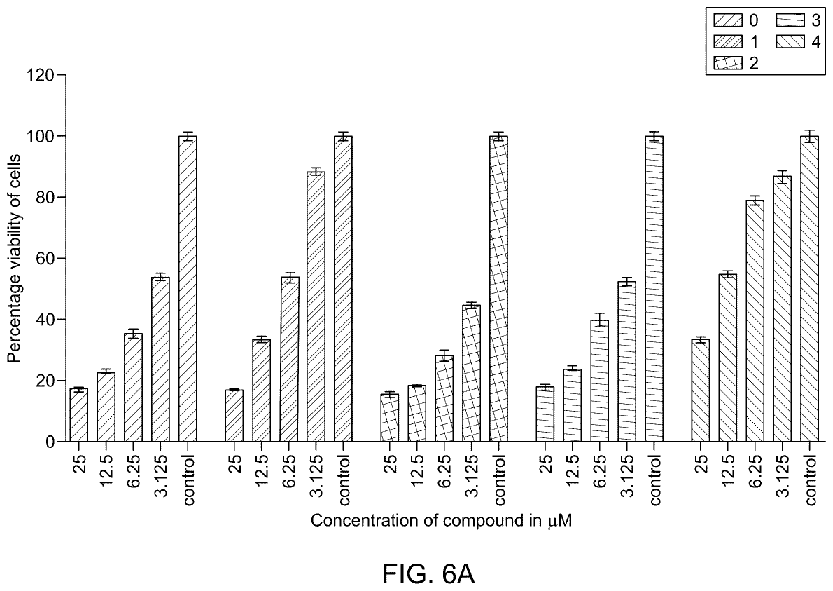

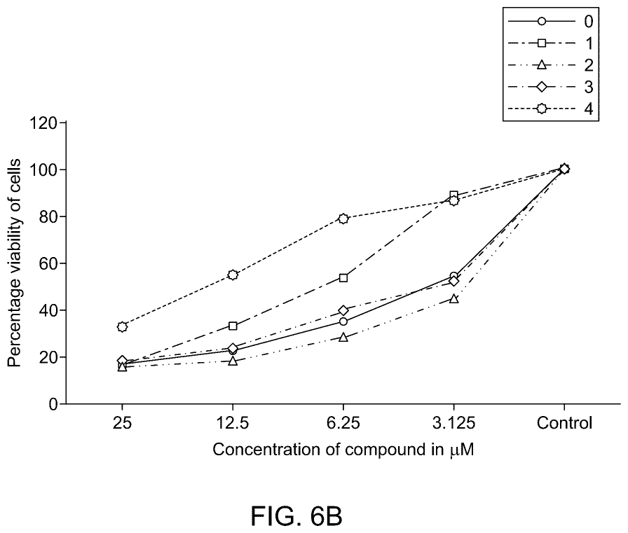

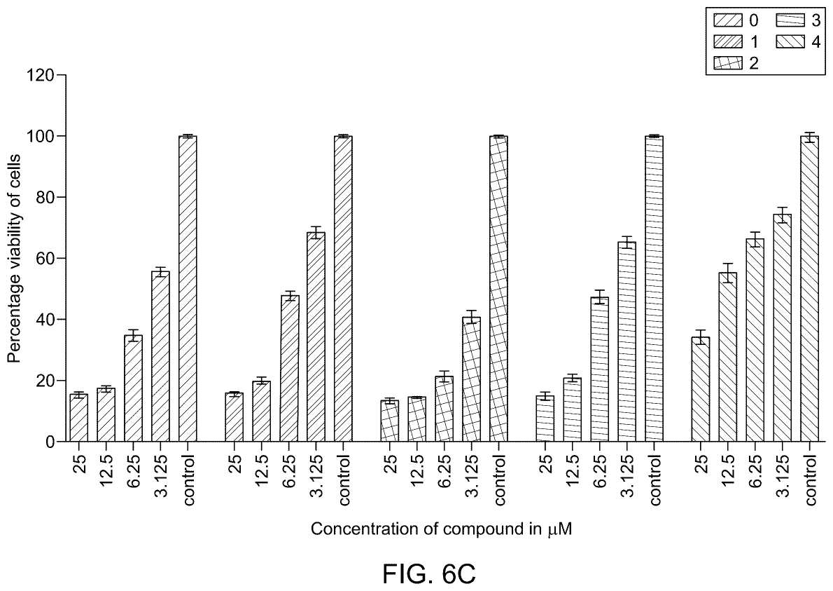

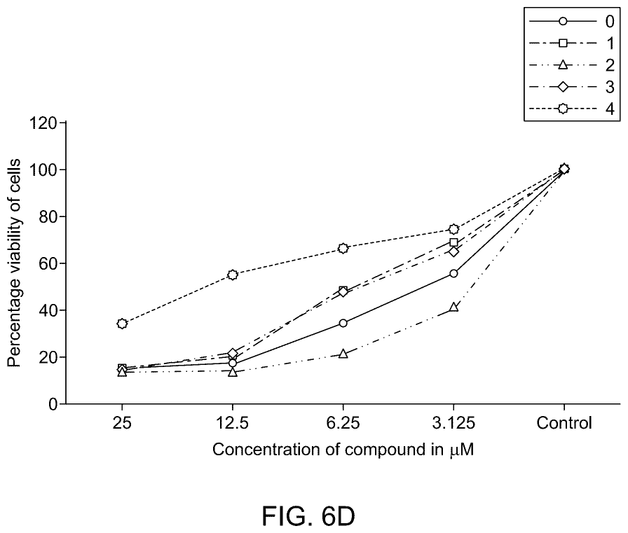

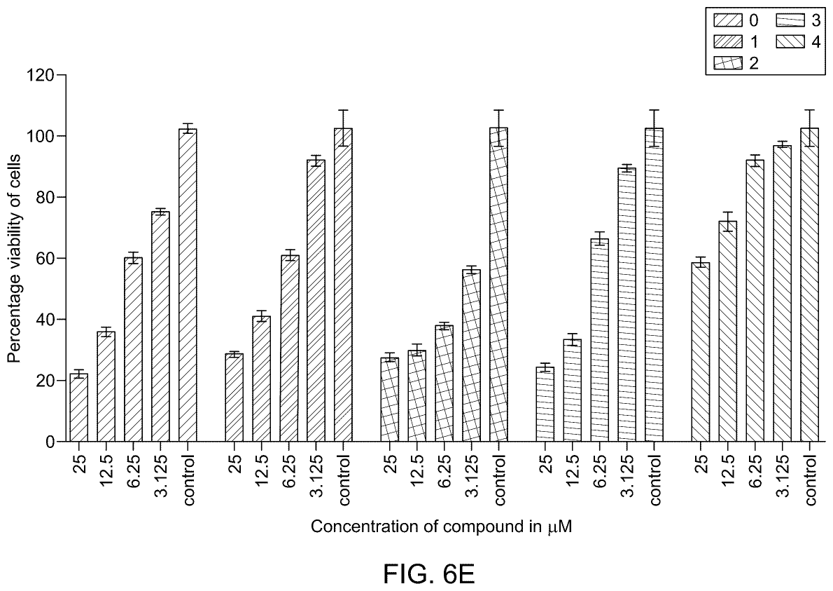

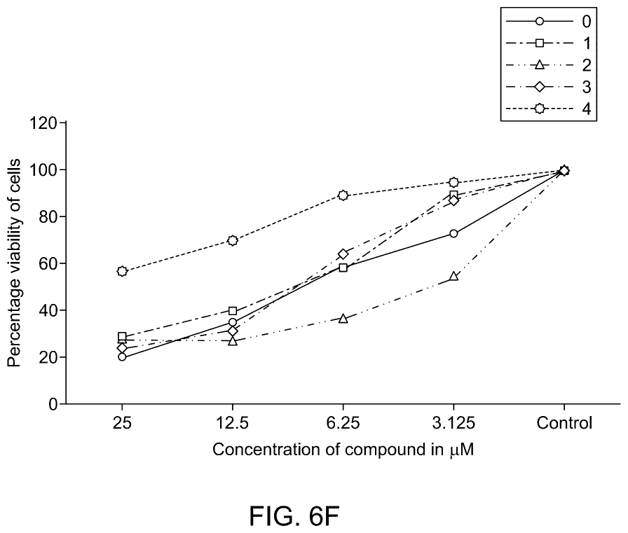

To evaluate the potential anticancer activity of the gold(J) complexes (0, 1-4), the gold(J) complexes (0, 1-4) were compared in vitro cytotoxicity to that of cisplatin (standard anticancer drug) in three cell lines derived from different human cancer lines including Human cervix cancer (Hela), Human osteosarcoma (MG-63) and Human colon cancer (HCT-15). The IC₅₀ values in concentration (M) obtained from the plot of the concentration of complexes against the percentage of cell viability are given in Table 7. Dose-dependent inhibition of cell proliferation was obtained by a specific increase in the concentration of cisplatin and gold complexes against a fixed number of three human cancer cell lines, as illustrated in FIGS. 6A-6F. The data in Table 7 shows that the gold complexes with lower IC₅₀ values were more effective than cisplatin in inhibiting the growth of cancer cells in all cases except complex 4 on the HeLa cell line is less effective with a higher IC₅₀ value (38.11 μM) than cisplatin (21.59 μM). However, complex 2 is the most active overall, 7 to 34 times more than cisplatin. Also, the complex [Au{(t-Bu)₃P}Cl] (0) was more effective than cisplatin. The greater cytotoxic activity of gold complexes indicates that the binding of more labile thione ligands has increased the inhibition efficiency of the gold(I) complex. This may be due to the ionic nature of the complex that favors its aqueous solubility.

The effectiveness of the complexes is different for the three cell lines.

TABLE 7
Half-maximal inhibitory concentrations (IC₅₀) of cisplatin
and new gold(I) complexes against HeLa, MG-63, and HCT-15
cancer cell lines. Values are mean (SD) expressed in μM.

| | Cell line | | | |
|---|---|---|---|---|
| Complex | | HeLa | MG-63 | HCT-15 |

| | | | | | | |
|---|---|---|---|---|---|---|
| Cisplatin | 21.59 | (0.73) | 33.37 | (1.2) | 31.94 | (0.55) |
| 0 | 7.9 | (0.25) | 3.40 | (0.25) | 3.26 | (0.22) |
| 1 | 10.15 | (0.28) | 5.68 | (0.18) | 8.37 | (0.05) |
| 2 | 3.24 | (0.26) | 1.03 | (0.32) | 1.66 | (0.14 |
| 3 | 9.44 | (0.04) | 5.40 | (0.27) | 3.54 | (0.15) |
| 4 | 38.11 | (2.28) | 13.50 | (1.3) | 14.70 | (0.25) |

### Example 9: Semi-Quantitative RT-PCR

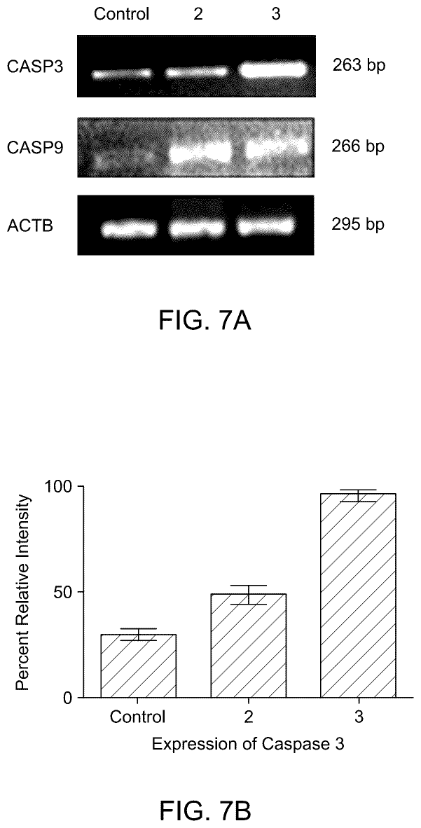

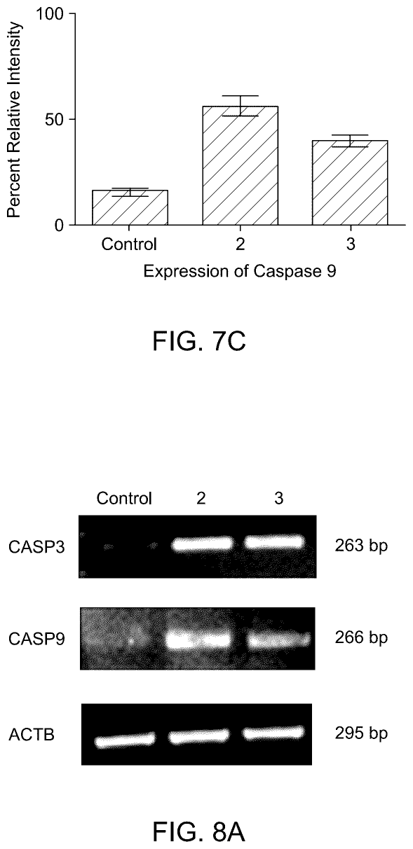

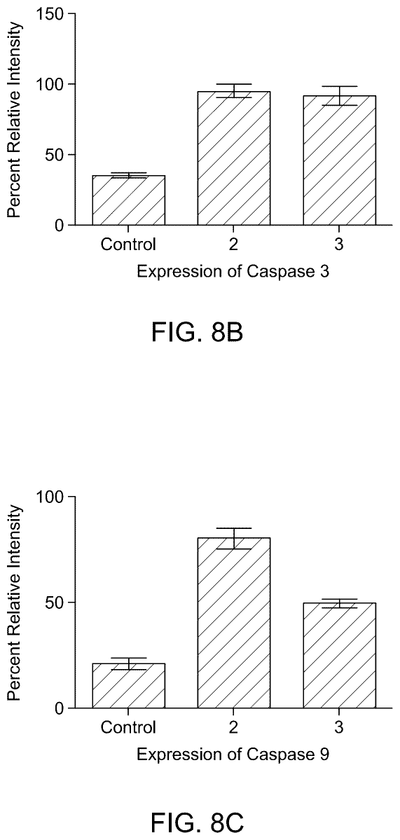

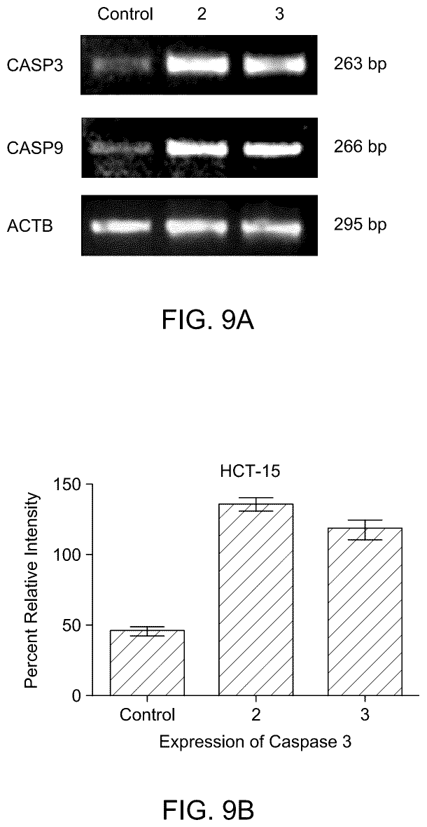

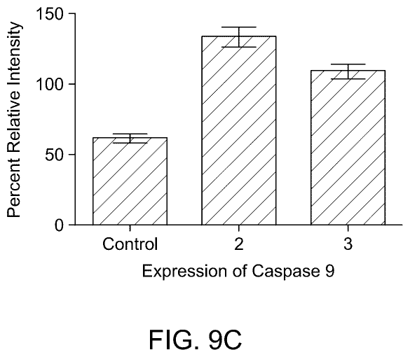

The toxic attribute of complexes 2 and 3 was further determined by studying the expression levels of caspase 3 and caspase 9 genes, the apoptotic markers at the mRNA level (FIGS. 7A-9C). The levels of both the caspases were measured as percent relative intensity expression to 3-actin of the respective groups. Compared to the control group, treatment of 2 and 3 with HeLa, MG-63, and HCT-15 cells induced apoptosis via an upregulation in the expression of both caspase 3 and 9 at mRNA level (p<0.005). The mRNA expressions of the caspases in 2 and 3 treated cells are represented in histograms. The results represent the apoptotic induction in all the selected cell lines with the treatment of complexes 2 and 3. The present results align with the earlier studies representing the toxic attribute of complexes by the up-regulated expression of caspase 3 and caspase-9.

### Example 10: In Vivo Animal Study

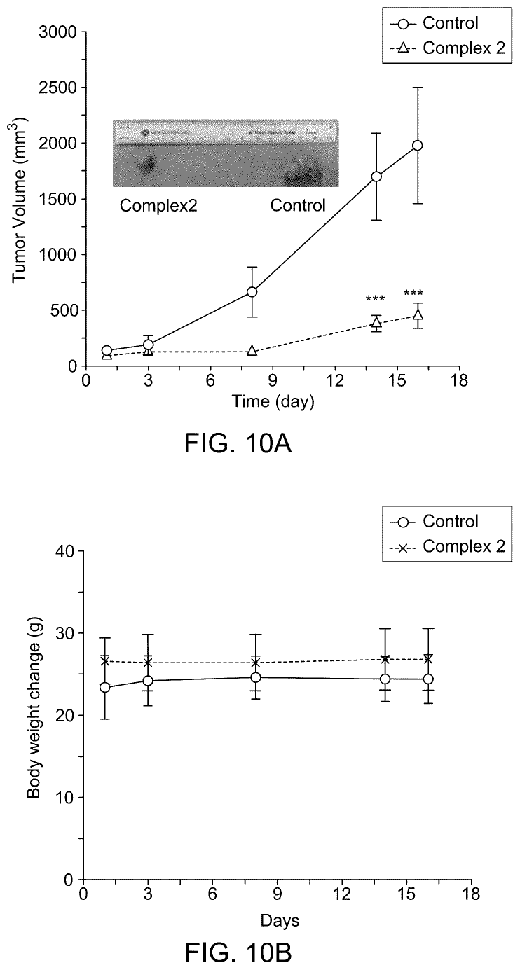

The anticancer effects of all complexes showed effects against the selected cancer cell lines, specifically complex 2. Therefore, complex 2 was subjected to further in vivo investigation using male nude mice bearing human colorectal cancer cell line HCT-116. The results showed that the control group had a rapid and sustained growth reaching 2000-millimetre cube (mm³) 18 days after xenograft. The tumor in mice treated with complex 2 showed a remarkable tumor suppression (FIG. 10A). In early post-transplant days, both groups of mice showed non-significant differences in tumor volume. Mice treated with Complex 2 (8 mg/kg dissolved in 5% DMSO in normal saline) once weekly showed a tumor volume reduction. The data is presented as mean±SEM. N>5 in each group (**p<0.01, TWO Way ANOVA followed by Sidak's test.

In addition, the changes in body weight were monitored as a preliminary indicator of treatment-induced toxicity. The results showed no difference in the body weight between the control and the complex 2 treated mice during the experiment (FIG. 10B). No change in body weight was observed during the treatment regimen and the control (P>0.05; Two-way ANOVA followed by Tukey's multiple comparison test). The present results demonstrated that complex 2 has a safe antitumor effect using animal models to provide clear insight into the effect of the synthesized complexes.

Numerous modifications and variations of the present disclosure are possible in light of the above teachings. It is therefore to be understood that within the scope of the appended claims, the invention may be practiced otherwise than as specifically described herein.

1. A method for treating a cancer, comprising:
administering to a subject in need thereof a complex of formula (I), 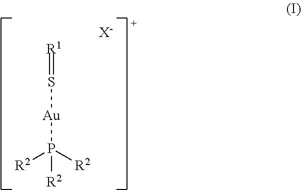
wherein R¹ is a substituted or unsubstituted cyclic amine,
wherein a ring of the cyclic amine has 5-7 atoms,
wherein R² is a straight or branched aliphatic carbon chain comprising 3-10 carbon atoms, and
wherein X is a counter ion.

2. The method of claim 1, wherein the ring of the cyclic amine has 6 atoms and has the structure of formula (II),

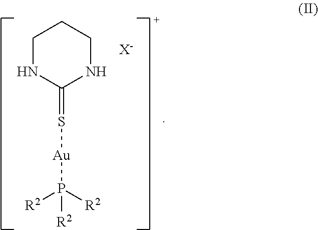

3. The method of claim 1, wherein the ring of the cyclic amine has 7 atoms and has the structure of formula (III),

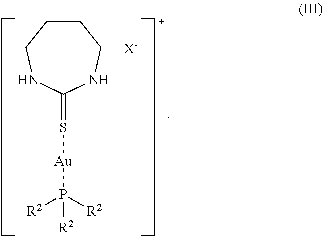

4. The method of claim 1, wherein the cyclic amine is substituted with a substituted or unsubstituted aliphatic chain comprising 1-10 carbon atoms at a nitrogen position of the cyclic amine.

5. The method of claim 1, wherein the cyclic amine is substituted with an unsubstituted aliphatic chain comprising 2 carbon atoms at a nitrogen position of the cyclic amine and has a structure of formula (IV),

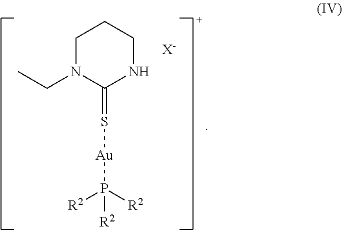

6. The method of claim 1, wherein the cyclic amine is substituted with a substituted aliphatic chain comprising 2 carbon atoms and a terminal alcohol at a nitrogen position of the cyclic amine and has a structure of formula (V),

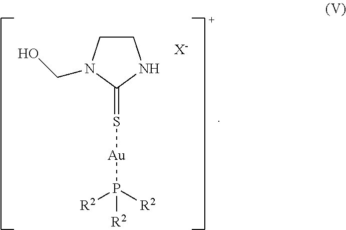

7. The method of claim 1, wherein the counter ion is selected from the group consisting of fluoride, chloride, iodide, triflate, and hexafluorophosphate (PF₆⁻).

8. The method of claim 1, wherein R² is a tert-butyl group.

9. The method of claim 1, wherein the cancer is cervical cancer, osteosarcoma, or colon cancer.

10. The method of claim 1, wherein the complex has a IC₅₀ of 1-15 micromolar (μM) against colon cancer cells in the subject.

11. The method of claim 1, wherein the complex has a IC₅₀ of 3-40 μM against cervical cancer cells in the subject.

12. The method of claim 1, wherein the complex has a IC₅₀ of 0.1-15 μM against osteosarcoma cancer cells in the subject.

13. The method of claim 1, wherein following the administering an amount of colon cancer cells in the subject are reduced by at least 50%.

14. The method of claim 1, wherein following the administering an amount of cervical cancer cells in the subject are reduced by at least 40%.

15. The method of claim 1, wherein following the administering an amount of osteosarcoma cancer cells in the subject are reduced by at least 60%.

16. The method of claim 1, wherein the administering is 1-20 milligrams (mg) of the complex per kilogram (kg) of the subject.

17. The method of claim 1, wherein the administering is by oral administration, parenteral administration, topical application, transdermally or nasally.

18. The method of claim 1, wherein the complex is 5-30 times more effective at reducing a number of cancer cells in the subject than cisplatin.

19. The method of claim 2, wherein following the administering an amount of colon cancer cells or osteosarcoma cancer cells in the subject are reduced by at least 80%.
